SEQUENCE LISTING (10 sequences)

SEQ ID NO: 1 - protein, 106 aa, Homo Sapiens
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGA

SEQ ID NO: 2 - protein, 227 aa, Homo Sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 345 aa, Homo Sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 11 aa, Homo Sapiens
GTELSVRAKPS

SEQ ID NO: 5 - protein, 118 aa, Homo Sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 6 - protein, 229 aa, Homo Sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 229 aa, Homo Sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 345 aa, Homo Sapiens; identical to SEQ ID NO: 3

SEQ ID NO: 9 - protein, 347 aa, Homo Sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 5 aa, Artificial Sequence
  Synthetic Polypeptide
  REPEAT (1)..(5)
GGGGS

(19) United States
(12) Patent Application Publication
(10) Pub. No.: US 2020/0157179 A1
(43) Pub. Date: May 21, 2020

(54) CD47 BLOCKADE THERAPY

(71) Applicant: TRILLIUM THERAPEUTICS INC., Mississauga (CA)

(72) Inventors: Gloria Hoi Ying LIN, Oakville (CA); Natasja Nielsen VILLER, Oakville (CA); Lisa Danae Schultz JOHNSON, Etobicoke (CA); Mark Michael WONG, Toronto (CA); Robert Adam UGER, Richmond Hill (CA)

(73) Assignee: Trillium Therapeutics Inc., Mississauga, ON (CA)

(21) Appl. No.: 16/496,168

(22) PCT Filed: Mar. 27, 2018
(86) PCT No.: PCT/CA2018/050368

§ 371 Date: Sep. 20, 2019

Related U.S. Application Data
(60) Provisional application No. 62/477,612, filed on Mar. 28, 2017.

(51) Int. Cl. C07K 14/705; C07K 16/28; A61K 38/00; A61K 47/68

(52) U.S. Cl. CPC C07K 14/70596; C07K 16/2803; A61K 47/68; C07K 2317/74; A61K 38/00; C07K 2319/30

(57) ABSTRACT

CD47+ disease cells, such as various cancers, are treated using a combination of CD47 blockade with T cell checkpoint inhibition. Preferred embodiments use SIRPαFc in combination with a PD-1 pathway inhibitor such as nivolumab and/or a CTLA-4 inhibitor such as ipilimumab.

### FIELD OF THE INVENTION

This invention relates to methods of using a drug that blocks the CD47/SIRPα interaction. More particularly, the invention relates to methods and means that, in combination, are useful for improving cancer therapy.

### BACKGROUND TO THE INVENTION

Cancer cells are targeted for destruction by antibodies that bind to cancer cell antigens, and through recruitment and activation of macrophages by way of Fc receptor binding to the Fc portion of that antibody. Binding between CD47 on cancer cells and SIRPα on macrophages transmits a “don't eat me” signal that enables many tumour cells to escape destruction by macrophages. It has been suggested that inhibition of the CD47/SIRPα interaction (CD47 blockade) will allow macrophages to “see” and destroy the target CD47+ cancer cell. The use of SIRPα to treat cancer by CD47 blockade is described in WO2010/130053.

In WO2014/094122, we describe a drug that inhibits the interaction between CD47 and SIRPα. This CD47 blockage drug is a form of human SIRPα that incorporates a particular region of its extracellular domain linked with a particularly useful form of an IgG1-based Fc region. In this form, the SIRPαFc drug shows dramatic effects on the viability of cancer cells that present with a CD47+ phenotype. The effect is seen particularly on acute myelogenous leukemia (AML) cells, and on many other types of cancer. A soluble form of SIRP having significantly altered primary structure and enhanced CD47 binding affinity is described in Stanford's WO2013/109752.

Other CD47 blockade drugs have been described in the literature and these include various CD47 antibodies (see for instance Stanford's U.S. Pat. No. 8,562,997, and InhibRx' WO2014/123580), each comprising different antigen binding sites but having, in common, the ability to compete with endogenous SIRPα for binding to CD47, thereby to allow interaction with macrophages and, ultimately, an increase in the rate of CD47+ cancer cell depletion. These drugs, while having a CD47 blockade effect, show activities in vivo that are quite different from those displayed by SIRPαFc-based drugs. The latter, for instance, display negligible binding to red blood cells whereas the opposite property in CD47 antibodies creates a need for dosing strategies that accommodate the drug “sink” that follows administration.

Still other agents are proposed for use in blocking the CD47/SIRPα axis. These include CD47Fc proteins (see Viral Logic's WO2010/083253), and SIRPα antibodies as described in UHN's WO2013/056352, in Stanford's WO2016/022971, in Eberhard's U.S. Pat. No. 6,913,894, and elsewhere.

The CD47 blockade approach in anti-cancer drug development shows great promise. It would be useful to provide methods and means for improving the effect of these drugs, and in particular for improving the effect of the CD47 blockade drug forms, especially those that incorporate SIRPα.

### SUMMARY OF THE INVENTION

It is now found that the anti-cancer effect of a SIRPα-based CD47 blockade drug is improved when combined with an agent that inhibits a T cell checkpoint, such as agents that inhibit the programmed death-1 (PD-1) and CTLA4 pathways. The invention includes a variety of methods/uses, materials, compositions, combinations, kits, and other articles of manufacture relating to this finding. In embodiments, the T cell checkpoint inhibitor is a CTLA-4 inhibitor or an antagonist that binds to PD-1, or that binds to a binding partner of PD-1, such as PD-L1 or PD-L2. In some embodiments, the CD47 blockade drug is a SIRPα-Fc. The two drugs cooperate in their effects on cancer cells, and result in the depletion of more cancer cells than can be accounted for by SIRPαFc alone.

In one aspect, there is provided a method for treating a subject presenting with CD47+ cancer cells, comprising administering to the subject a SIRPα-Fc drug, and a T cell checkpoint inhibitor such as a PD-1 blockade drug and/or a CTLA-4 inhibitor. The term SIRPα-Fc (or SIRPαFc) refers to a genus of drugs comprised of a SIRPα domain attached directly or indirectly to an Fc domain. The SIRPα domain is derived from human SIRPα and includes sufficient SIRPα structure to retain CD47-binding activity characteristic of SIRPα, but is soluble and lacks at least the transmembrane domain of SIRPα encoded by the genome. An exemplary SIRPα domain comprises the IgV domain as described below. The Fc domain has the characteristics of an antibody constant region, as described below in greater detail.

In a related aspect, the present invention provides for the use of a SIRPα-Fc drug in combination with a T cell checkpoint inhibitor such as a PD-1 pathway inhibitor and/or a CTLA-4 inhibitor, for the treatment of a subject presenting with a CD47+ cancer.

There is also provided, in another aspect, a pharmaceutical combination of anti-cancer drugs comprising SIRPαFc and a T cell checkpoint inhibitor such as a PD-1 blockade drug and/or a CTLA-4 inhibitor, together with instructions teaching their use in the treatment method herein described. Thus, there is provided a pharmaceutical combination that comprises a SIRPαFc and at least one T cell checkpoint inhibitor, wherein the T cell checkpoint inhibitor can be nivolumab or ipilimumab. In another related aspect, the pharmaceutical combination comprises a SIRPαFc and at least two T cell checkpoint inhibitors that are nivolumab and ipilimumab. The three drugs cooperate in their effects on increasing anti-tumor immune response, resulting in depletion of more cancer cells than can be accounted for by SIRPαFc alone.

The invention further includes methods, uses, products and compositions as summarized in the following numbered paragraphs:
1. A method for treating a subject presenting with CD47+ disease cells, comprising administering to the subject a T cell checkpoint inhibitor and a CD47 blockade drug.
2. The use of a T cell checkpoint inhibitor and a CD47 blockade drug to treat a subject presenting with CD47+ disease cells.
3. A T cell checkpoint inhibitor for use in treating CD47+ disease cells by co-administration with a CD47 blockade drug.
4. A CD47 blockade drug for use in treating CD47+ disease cells by co-administration with a T cell checkpoint inhibitor.
5. The use of a T cell checkpoint inhibitor and a CD47 blockade drug in the manufacture of a medicament for treating CD47+ disease cells.
6. The use of a T cell checkpoint inhibitor in the manufacture of a medicament for treating CD47+ disease cells by co-administration with a CD47 blockade drug.
7. The use of a CD47 blockade drug in the manufacture of a medicament for treating CD47+ disease cells by co-administration with a T cell checkpoint inhibitor.
8. A product comprising a T cell checkpoint inhibitor and a CD47 blockade drug as a combined preparation for simultaneous, separate, or sequential use in the treatment of CD47+ disease cells.
9. A composition comprising a T cell checkpoint inhibitor, a CD47 blockade drug, and a pharmaceutically acceptable carrier.
10. The method, use, product, or composition according to any one of paragraphs 1-9, wherein the CD47+ disease cells comprise CD47+ cancer cells.
11. The method, use, product, or composition according to any one of paragraphs 1-9, wherein the T cell checkpoint inhibitor comprises a PD-1 blockade drug.
12. The method, use, product, or composition according to paragraph 11, wherein the PD-1 blockade drug comprises an agent that binds PD-1.
13. The method, use, product, or composition according to paragraph 12, wherein the PD-1 blockade drug comprises nivolumab.
14. The method, use, product, or composition according to any one of paragraphs 1-13, wherein the PD-1 blockade drug comprises an agent that binds PD-L1 or PD-L2.
15. The method, use, product, or composition according to paragraph 14, wherein the PD-1 blockade drug comprises a PD-L1 binding agent.
16. The method, use, product, or composition according to paragraph 15, wherein the PD-L1 binding agent comprises a member selected from durvalumab, atezolizumab, avelumab and the IgG4 antibody designated BMS-936559/MDX1105
17. The method, use, product, or composition according to any one of paragraphs 1-16, wherein the T cell checkpoint inhibitor comprises a CTLA4 inhibitor.
18. The method, use, product, or composition according to paragraph 17, wherein the CTLA4 inhibitor comprises a CTLA4 antibody.
19. The method, use, product, or composition according to paragraph 18, wherein the CTLA4 antibody comprises ipilimumab or tremelimumab.
20. The method, use, product, or composition according to any one of paragraphs 1-19, wherein the CD47 blockade drug comprises an Fc fusion protein comprising a soluble CD47-binding region of human SIRPα fused to an Fc region of an antibody.
21. The method, use, product, or composition according to paragraph 20, wherein the Fc fusion protein comprising soluble SIRPα comprises the amino acid sequence of SEQ ID NO: 8.
22. The method, use, product, or composition according to paragraph 20, wherein the Fc fusion protein comprising soluble SIRPα comprises the amino acid sequence of SEQ ID NO: 9.
23. The method, use, product, or composition according to any one of paragraphs 1-19, wherein the CD47 blockade drug comprises soluble SIRPα having one or more amino acid substitutions selected from L⁴V/I, V⁶I/L, A²¹V, V²⁷I/L, I³¹T/S/F, E⁴⁷V/L, K⁵³R, E⁵⁴Q, H⁵⁶P/R, S⁶⁶T/G, K⁶⁸R, V⁹²I, F⁹⁴V/L, V⁶³I, and F¹⁰³V.
24. The method, use, product, or composition according to any one of paragraphs 1-23, wherein the T cell checkpoint inhibitor comprises a combination of nivolumab and ipilimumab.
25. The method, use, product, or composition according to any one of paragraphs 1-24, wherein the CD47+ disease cells comprise blood cancer cells or solid tumour cancer cells.
26. The method, use, product, or composition according to paragraph 25, wherein the CD47+ disease cells are cells of a cancer type selected from acute lymphocytic leukemia (ALL); acute myeloid leukemia (AML); chronic lymphocytic leukemia (CLL); chronic myelogenous leukemia (CML); myeloproliferative disorder/neoplasm (MPDS); and myelodysplastic syndrome.
27. The method, use, product, or composition according to paragraph 25, wherein the cancer is a lymphoma selected from a Hodgkin's lymphoma, both indolent and aggressive non-Hodgkin's lymphoma, Burkitt's lymphoma, and follicular lymphoma (small cell and large cell).
28. The method, use, product, or composition according to paragraph 25, wherein the cancer is a myeloma selected from multiple myeloma (MM), giant cell myeloma, heavy-chain myeloma, and light chain or Bence-Jones myeloma.
29. The method, use, product, or composition according to paragraph 25, wherein the cancer is a melanoma.
30. The method, use, product, or composition according to paragraph 25, wherein the cancer is AML, myelodysplastic syndrome, CLL, Hodgkin lymphoma, indolent B cell lymphoma, aggressive B cell lymphoma, T cell lymphoma, multiple myeloma, myeloproliferative neoplasms, or CD20+ lymphoma.
31. The method, use, product, or composition according to paragraph 25, wherein the cancer is selected from non-small cell lung cancer, renal cancer, bladder cancer, head and neck squamous cell carcinoma, Merkel cell skin cancer, esophageal cancer, pancreatic cancer, hepatocellular carcinoma, glioblastoma, gastric cancer, breast cancer and ovarian cancer.
32. The method, use, product, or composition according to paragraph 25, wherein the cancer is selected from melanoma, metastatic non-small cell lung cancer, head and neck cancer, Hodgkin's lymphoma, urothelial carcinoma and gastric cancer.
33. The method, use, product, or composition according to any one of paragraphs 1-32, wherein the T cell checkpoint inhibitor and the CD47 blockade drug are present or used in synergistically effective amounts.
34. A pharmaceutical combination of anti-cancer agents, comprising a SIRPαFc and a T cell checkpoint inhibitor effective to enhance SIRPαFc-mediated depletion of CD47+ disease cells.
35. The use of the combination according to paragraph 34, for the treatment of a subject presenting with CD47+ cancer cells.
36. The use according to paragraph 35, wherein the CD47+ disease cells are CD47+ cancer cells. 37. A kit comprising at least one of SIRPαFc and a T cell checkpoint inhibitor, and instructions teaching the use thereof according to the method, use, product, or composition of any one of paragraphs 1-33.

Aspects of the invention that have been described herein as methods also can be described as “uses,” and all such uses are contemplated as aspects of the invention. Likewise, compositions described herein as having a “use” can alternatively be described as processes or methods of using, which are contemplated as aspects of the invention.

Likewise, details of the invention that are described herein in relation to a particular method, use, composition, or other product should be understood to be applicable to other aspects or embodiments of the invention, including aspects or embodiments considered to be different classes of invention for examination or other purposes.

The invention includes, as an additional aspect, all embodiments of the invention narrower in scope in any way than the variations defined by specific paragraphs above. For example, where certain aspects of the invention that are described as a genus or set, it should be understood that every member of a genus or set is, individually, an aspect of the invention. Likewise, every individual subset is intended as an aspect of the invention. By way of example, if an aspect of the invention is described as a members selected from the group consisting of 1, 2, 3, and 4, then each individual subgroup (e.g., members selected from {1,2,3}or {1,2,4} or{2,3,4} or {1,2} or {1,3} or {1,4} or {2,3} or {2,4} or {3,4}) and each individual species {1} or {2} or {3} or {4} is contemplated as an aspect or variation of the invention. Likewise, if an aspect of the invention is characterized as a range, such as a temperature range, then integer sub-ranges are contemplated as aspects or variations of the invention.

The headings herein are for the convenience of the reader and not intended to be limiting. Additional aspects, embodiments, and variations of the invention will be apparent from the Detailed Description and/or Drawing and/or claims.

Although the Applicant invented the full scope of the invention described herein, the Applicant does not intend to claim subject matter described in the prior art work of others. Therefore, in the event that statutory prior art within the scope of a claim is brought to the attention of the Applicant by a Patent Office or other entity or individual, the Applicant reserves the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

These and other aspects of the invention are now described in greater detail with reference to the accompanying drawings, in which:

### BRIEF REFERENCE TO THE DRAWINGS

FIG. 1 shows the drug combination study design and the dosing regimen.

FIG. 2 shows tumour volumes at various time points (in days) following monotherapy, combination therapy and control dosing.

FIG. 3 provides survival curves (Kaplan-Meier plots) after 60 days from initial treatment. One animal in the anti-PD-1 monotherapy group and in the anti-PD-1 plus SIRPαFc combination group died before reaching tumour endpoint.

FIG. 4 shows the effect of the combinations of nivolumab and/or ipilimumab with SIRPαFc in modulation of tumor specific CD8+ T cell activation and effector functions in vitro, as measured by the percentage of CD107a/b+ and TNFα+ IFNγ+.

### DETAILED DESCRIPTION AND PREFERRED EMBODIMENTS

The present invention provides an improved method for treating subjects presenting with cancer cells and tumours that have a CD47+ phenotype. In this method, subjects receive a combination of SIRPαFc, as a CD47 blockade drug, and a T cell checkpoint inhibitor, such as a PD-1 blockade drug.

In some variations, the subjects received the two (or more) agents to produce a synergistic effect. In the context of administration of two or more agents, “synergistically effective amounts” are amounts of the agents that either (i) produce greater than additive therapeutic effects, compared to monotherapy with the agents; or (ii) produce at least comparable therapeutic effects and reduce toxic side effects, due to lower effective dosing or less frequent dosing, compared to monotherapy with one of the agents. An indication of such synergy can be provided in in vitro studies, e.g., with cell lines, in studies to evaluate the killing of tumor cell lines. Synergy can be demonstrated in clinical trials in which the effects of monotherapy and combination therapy are compared and statistically analyzed.

A “blockade drug” is also referred to herein as a “blocking agent”.

The SIRPαFc used in the present method is a monomeric or homodimeric or heterodimeric form of a single chain polypeptide comprising an Fc region (or fragment) of an antibody and a CD47-binding region (or fragment) of human SIRPα. Soluble SIRPα-based drugs of this general type are described in the literature and include those referenced in University Health Network's International Patent Application No. PCT/CA2008/001814, published as WO 2009/046541; Novartis' Patent Application No. PCT/EP2009/067411, published as WO 2010/070047; Stanford's Patent Application No. PCT/US2013/021937, published as WO2013/109752; and Trillium Therapeutic's Patent Application No. PCT/CA2013/001046, published as WO2014/094122, all incorporated herein by reference in their entirety and specifically for their descriptions of SIRPα-based constructs.

In preferred embodiments, the SIRPαFc has the properties discussed below. More particularly, the drug suitably comprises the human SIRPα protein, in a form fused directly, or indirectly, with an antibody constant region, or Fc (fragment crystallisable) Unless otherwise stated, the term “human SIRPα” as used herein refers to a wild type, endogenous, mature form of human SIRPα. In humans, the SIRPα protein is found in two major forms. One form, the variant 1 or V1 form, has the amino acid sequence set out as NCBI RefSeq NP_542970.1 (residues 27-504 constitute the mature form). Another form, the variant 2 or V2 form, differs by 13 amino acids and has the amino acid sequence set out in GenBank as CAA71403.1 (residues 30-504 constitute the mature form). These two forms of SIRPα constitute about 80% of the forms of SIRPα present in humans, and both are embraced herein by the term “human SIRPα”. Also embraced by the term “human SIRPα” are the minor forms thereof that are endogenous to humans and have the same property of triggering signal transduction through CD47, upon binding thereto. The present invention is directed most particularly to the drug combinations that include the variant 2 form, or V2.

In the present drug combination, useful SIRPαFc fusion proteins comprise one of the three so-called immunoglobulin (Ig) domains that lie within the extracellular region of human SIRPα. More particularly, the present SIRPαFc proteins incorporate residues 32-137 of human SIRPα (a 106-mer), which constitute and define the IgV domain of the V2 form according to current nomenclature. This SIRPα sequence, shown below, is referenced herein as SEQ ID NO: 1.

(SEQ ID NO: 1)
EELQVIQPDKSVSVAAGESAILHCTVTSLIPVGPIQWFRGAGPARELIY
NQKEGHFPRVTTVSESTKRENMDFSISISNITPADAGTYYCVKFRKGSP
DTEFKSGA

In a preferred embodiment, the SIRPαFc fusion protein incorporates the IgV domain as defined by SEQ ID NO: 1, and additional, flanking residues contiguous within the wild type human SIRPα sequence. This preferred form of the IgV domain, represented by residues 31-148 of the V2 form of human SIRPα, is a 118-mer having SEQ ID NO: 5 shown below:

(SEQ ID NO: 5)
EEELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVRAKPS

The Fc region of the SIRPαFc fusion preferably does have effector function. Fc refers to “fragment crystallisable” and represents the constant region of an antibody comprised principally of the heavy chain constant region and components within the hinge region. Suitable Fc components thus are those having effector function. An Fc component “having effector function” is an Fc component having at least some effector function, such as at least some contribution to antibody-dependent cellular cytotoxicity or some ability to fix complement. Also, the Fc will at least bind to one or more types of Fc receptor. These properties can be revealed using assays established for this purpose. Functional assays include the standard chromium release assay that detects target cell lysis. By this definition, an Fc region that is wild type IgG1 or IgG4 has effector function, whereas the Fc region of a human IgG4 mutated to eliminate effector function, such as by incorporation of an alteration series that includes Pro233, Va1234, Ala235 and deletion of Gly236 (EU), is considered not to have effector function. In a preferred embodiment, the Fc is based on human antibodies of the IgG1 isotype. The Fc region of these antibodies will be readily identifiable to those skilled in the art. In embodiments, the Fc region includes the lower hinge-CH2-CH3 domains.

In a specific embodiment, the Fc region is based on the amino acid sequence of a human IgG1 set out as P01857 in UniProtKB/Swiss-Prot, residues 104-330, and has the amino acid sequence shown below and referenced herein as SEQ ID NO: 2:

(SEQ ID NO: 2)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK*

Thus, in embodiments, the Fc region has either a wild type or consensus sequence of an IgG1 constant region. In alternative embodiments, the Fc region incorporated in the fusion protein is derived from any IgG1 antibody having a typical effector-active constant region. The sequences of such Fc regions can correspond, for example, with the Fc regions of any of the following IgG1 sequences (all referenced from GenBank), for example: BAG65283 (residues 242-473), BAC04226.1 (residues 247-478), BAC05014.1 (residues 240-471), CAC20454.1 (residues 99-320), BAC05016.1 (residues 238-469), BAC85350.1 (residues 243-474), BAC85529.1 (residues 244-475), and BAC85429.1 (residues (238-469).

In other embodiments, the Fc region has a sequence of a wild type human IgG4 constant region. In alternative embodiments, the Fc region incorporated in the fusion protein is derived from any IgG4 antibody having a constant region with effector activity that is present but, naturally, is significantly less potent than the IgG1 Fc region. The sequences of such Fc regions can correspond, for example, with the Fc regions of any of the following IgG4 sequences: P01861 (residues 99-327) from UniProtKB/Swiss-Prot and CAC20457.1 (residues 99-327) from GenBank.

In a specific embodiment, the Fc region is based on the amino acid sequence of a human IgG4 set out as P01861 in UniProtKB/Swiss-Prot, residues 99-327, and has the amino acid sequence shown below and referenced herein as SEQ ID NO: 6:

(SEQ ID NO: 6)
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

In embodiments, the Fc region incorporates one or more alterations, usually not more than about 5 such alterations, including amino acid substitutions that affect certain Fc properties. In one specific and preferred embodiment, the Fc region incorporates an alteration at position 228 (EU numbering), in which the serine at this position is substituted by a proline (S²²⁸P), thereby to stabilize the disulfide linkage within the Fc dimer. Other alterations within the Fc region can include substitutions that alter glycosylation, such as substitution of Asn²⁹⁷ by glycine or alanine; half-life enhancing alterations such as T²⁵²L, T²⁵³S, and T²⁵⁶F as taught in U.S. 62/777,375, and many others. Particularly useful are those alterations that enhance Fc properties while remaining silent with respect to conformation, e.g., retaining Fc receptor binding.

In a specific embodiment, and in the case where the Fc component is an IgG4 Fc, the Fc incorporates at least the S²²⁸P mutation, and has the amino acid sequence set out below and referenced herein as SEQ ID NO: 7:

(SEQ ID NO: 7)
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

The CD47 blockade drug used in the combination is thus a SIRPαFc fusion protein useful to inhibit binding between human SIRPα and human CD47, thereby to inhibit or reduce transmission of the signal mediated via SIRPα-bound CD47, the fusion protein comprising a human SIRPα component and, fused therewith, an Fc component, wherein the SIRPα component comprises or consists of a single IgV domain of human SIRPα V2 and the Fc component is the constant region of a human IgG, wherein the constant region preferably has effector function.

In one embodiment, the fusion protein comprises a SIRPα component consisting at least of residues 32-137 of the V2 form of wild type human SIRPα, i.e., SEQ ID NO: 1. In a preferred embodiment, the SIRPα component consists of residues 31-148 of the V2 form of human SIRPα, i.e., SEQ ID NO: 5. In another embodiment, the Fc component is the Fc component of the human IgG1 designated P01857, and in a specific embodiment has the amino acid sequence that incorporates the lower hinge-CH2-CH3 region thereof i.e., SEQ ID NO: 2.

In a preferred embodiment, therefore, the present invention provides a SIRPαFc fusion protein, as both an expressed single chain polypeptide and as a secreted dimeric fusion thereof, wherein the fusion protein incorporates a SIRPα component having SEQ ID NO: 1 and preferably SEQ ID NO: 5 and, fused therewith, an Fc region having effector function and having SEQ ID NO: 2. When the SIRPα component is SEQ ID NO: 1, this fusion protein comprises SEQ ID NO: 3, shown below:

(SEQ ID NO: 3)
EEELQVIQPDKSVSVAAGESAILHCTVTSLIPVGPIQWERGAGPAREDY
NQKEGHFPRVTTVSESTKRENMDFSISISNITPADAGTYYCVKFRKGSP
DTEFKSGAGTELSVRAKPSDKTHTCPPCPAPELLGGPSVFLEPPKPKDT
LMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K*

When the SIRPα component is SEQ ID NO: 5, this fusion protein comprises SEQ ID NO: 8, shown below:

(SEQ ID NO: 8)
EEELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVRAKPSDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPP
VLDSDGSFFLYSKLTVDKSRWQQGNVFSCSVMHEALHN
HYTQKSLSLSPGK

In alternative embodiments, the Fc component of the fusion protein is based on an IgG4, and preferably an IgG4 that incorporates the S²²⁸P mutation. In the case where the fusion protein incorporates the preferred SIRPα IgV domain of SEQ ID NO: 5, the resulting IgG4-based SIRPα-Fc protein has SEQ ID NO: 9, shown below:

(SEQ ID NO: 9)
EEELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVRAKPSESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK

In preferred embodiment, the fusion protein comprises, as the SIRPα IgV domain of the fusion protein, a sequence that is SEQ ID NO: 5. The preferred SIRPαFc is SEQ ID NO: 8.

The SIRPα sequence incorporated within the CD47 blockade drug can be varied, as described in the literature. That is, useful substitutions within SIRPα include one or more of the following (relative to SEQ ID NO: 5, for example): L⁴V/I, V⁶I/L, A²¹V, V²⁷I/L, I³¹T/S/F, E⁴⁷V/L, K⁵³R, E⁵⁴Q, H⁵⁶P/R, S⁶⁶T/G, K⁶⁸R, V⁹²I, F⁹⁴V/L, V⁶³I, and/or F¹⁰³V. Still other substitutions include conservative amino acid substitutions in which an amino acid is replaced by an amino acid from the same group.

In the SIRPαFc fusion protein, the SIRPα component and the Fc component are fused, either directly or indirectly, to provide a single chain polypeptide that is ultimately produced as a dimer in which the single chain polypeptides are coupled through intrachain disulfide bonds formed between the Fc regions of individual single chain SIRPαFc polypeptides. The nature of the fusing region that joins the SIRPα region and the Fc is not critical. The fusion may be direct between the two components, with the SIRP component constituting the N-terminal end of the fusion and the Fc component constituting the C-terminal end. Alternatively, the fusion may be indirect, through a linker comprised of one or more amino acids, desirably genetically encoded amino acids, such as two, three, four, five, six, seven, eight, nine or ten amino acids, or any number of amino acids between 5 and 100 amino acids, such as between 5 and 50, 5 and 30 or 5 and 20 amino acids. A linker may comprise a peptide that is encoded by DNA constituting a restriction site, such as a BamHI, ClaI, EcoRI, HindIII, PstI, SalI and XhoI site and the like.

The linker amino acids typically and desirably will provide some flexibility to allow the Fc and the SIRPα components to adopt their active conformations. Residues that allow for such flexibility typically are Gly, Asn and Ser, so that virtually any combination of these residues (and particularly Gly and Ser) within a linker is likely to provide the desired linking effect. In one example, such a linker is based on the so-called G₄S sequence (Gly-Gly-Gly-Gly-Ser SEQ ID NO: 10) which may repeat as (G₄S)ₙ (SEQ ID NO: 10) where n is 1, 2, 3 or more, or is based on (Gly)n, (Ser)n, (Ser-Gly)n or (Gly-Ser)n and the like. In another embodiment, the linker is GTELSVRAKPS (SEQ ID NO: 4). This sequence constitutes SIRPα sequence that C-terminally flanks the IgV domain (it being understood that this flanking sequence could be considered either a linker or a different form of the IgV domain when coupled with the IgV minimal sequence described above). It is necessary only that the fusing region or linker permits the components to adopt their active conformations, and this can be achieved by any form of linker useful in the art.

The SIRPαFc fusion is useful to inhibit interaction between SIRPα and CD47, thereby to block signalling across this axis. Stimulation of SIRPα on macrophages by CD47 is known to inhibit macrophage-mediated phagocytosis by deactivating myosin-II and the contractile cytoskeletal activity involved in pulling a target into a macrophage. Activation of this cascade is therefore important for the survival of CD47+ disease cells. Blocking this pathway allows macrophages to engulf and eradicate the CD47+ disease cell population.

The term “CD47+” is used with reference to the phenotype of cells targeted for binding by the present polypeptides. The protein CD47, also known as integrin associated protein (IAP), is a transmembrane protein encoded by the CD47 gene. CD47 belongs to the immunoglobulin superfamily and interacts with, for example, membrane integrins, thrombospondin-1 (TSP-1), and signal-regulatory protein alpha (SIRPα). Cells that are CD47+ can be identified by flow cytometry using CD47 antibody as the affinity ligand. CD47 antibodies that are labeled appropriately are available commercially for this use (for example, clone B6H12 is available from Santa Cruz Biotechnology). The cells examined for CD47 phenotype can include standard tumour biopsy samples including particularly blood samples taken from the subject suspected of harbouring endogenous CD47+ cancer cells. CD47 disease cells of particular interest as targets for therapy with the present fusion proteins are those that “over-express” CD47. These CD47+ cells typically are disease cells, and present CD47 at a density on their surface that exceeds the normal CD47 density for a cell of a given type. CD47 overexpression will vary across different cell types, but is meant herein to refer to any CD47 level that is determined, for instance by flow cytometry as exemplified herein or by immunostaining or by gene expression analysis or the like, to be greater than the level measurable on a counterpart cell having a CD47 phenotype that is normal for that cell type.

The present drug combination comprises both SIRPαFc, as a CD47 blocking agent, and an immune cell checkpoint inhibitor. These include a wide variety of agents responsible for up--regulating the T-cell based immune system. Key inhibitors will block pathways that include CTLA4 and/or PD-1, and these inhibiting agents are embraced by the present invention in its broad context.

In specific embodiments, the immune cell checkpoint inhibitor is a PD-1 blockade drug, and these drugs block interaction between the PD-1 receptor and ligands such as PD-L1 and PD-L2.

PD-1 blockade drugs are used in combination with SIRPαFc in accordance with the present method. PD-1 itself (programmed death-1, aka CD279)) is a lymphocyte receptor that interacts with ligands designated PD-L1 and PD-L2. The PD-1 pathway lies within the B7:CD28 family that also comprises CTLA4 (aka CD152), and is involved in homeostasis of the immune system by controlling T cell activation. Expression of PD-1 ligand 1 (PD-L1, CD274, B7:H1)) and PD-1 ligand 2 (PD-L2, B7-DC, CD273) on cancer cells are negative prognostic factors. PD-1 blockade has been shown to be effective in treating a variety of cancer types.

The PD-1 blockade drugs (aka PD-1 pathway inhibitors) that can be used to block ligand-induced stimulation of PD-1 are numerous, and include Fc fusions and antibodies and their active fragments that bind PD-1 selectively, thereby to inhibit interaction with the ligands PD-L1 and PD-L2. Particularly useful PD-1/PD-L1 blockade drugs include fusion proteins and antibodies designated as pidilizumab, pembrolizumab, nivolumab, AMP-224 (a PD-L2-IgG2a Fc-fusion protein that targets PD-1), camrelizumab (SHR-1210), spartalizumab (PDR001) and REGM2810, and MEDI0680, a humanized IgG4.

The useful PD-1 blockade drugs also include agents that bind selectively to PD-L1 or PD-L2, particular PD-L1-binding forms of which include an IgG4 antibody known as BMS-936559 and IgG1 antibodies including durvalumab (MEDI4736), atezolizumab (MPDL3280A/RG7446) and avelumab (aka MSB001078C).

In a preferred embodiment, the T cell checkpoint inhibitor is a PD-1 blocking antibody that is nivolumab. Nivolumab is an approved human IgG4 antibody that binds human PD-1, and is sold under the name OPDIVO® (Bristol-Myers Squibb) for use as first line treatment for inoperable or metastatic melanoma in combination with ipilimumab.

The SIRPαFc drug combination can also include, instead of a PD-1 inhibitor or in combination therewith, any other immune checkpoint inhibitor including particularly a CTLA-4 inhibitor. Like PD-1, CTLA-4 negatively regulates T cell activation. The CTLA-4 inhibitors are those agents that block CTLA-4 from binding with the B7 ligand. CTLA-4 inhibitors are approved for human use, and these are useful in the present invention when combined with the CD47 blockade drug, SIRPαFc. Particularly useful CTLA-4 inhibitors and agents useful in the present invention are antibodies that bind CTLA4, including ipilimumab or tremelimumab and optionally belatacept, and abatacept.

In a preferred embodiment, the CTLA-4 inhibitor is ipilimumab. Ipilimumab is a fully human, recombinant antibody that binds T cell-expressed human CTLA-4 and has the trade name Yervoy® (Bristol-Myers Squibb). It is provided as an aqueous solution in 50 mg and 200 mg preservative-free, single-use vials in a concentration of 5 mg/mL.

In embodiments, the present drug combination comprises a combination of a SIRPαFc having SEQ ID NO: 8 or SEQ ID NO: 9 or SEQ ID NO: 7, and the PD-1 blockade antibody known as nivolumab. In a specific embodiment, the combination comprises SIRPαFc having SEQ ID NO: 8 and the antibody nivolumab. In another specific embodiment, the combination comprises SIRPαFc having SEQ ID NO: 9 and the antibody nivolumab. In these embodiments, the combination can further comprise the CTLA-4 inhibitor that is ipilimumab.

In other embodiments, the present drug combination comprises a combination of a SIRPαFc having SEQ ID NO: 8 or SEQ ID NO: 9 or SEQ ID NO: 7, and the CTLA-4 antibody known as ipilimumab. In a specific embodiment, the combination comprises SIRPαFc having SEQ ID NO: 8 and the antibody ipilimumab. In another specific embodiment, the combination comprises SIRPαFc having SEQ ID NO: 9 and the antibody ipilimumab. In these embodiments, the combination can further comprise the PD-1 antibody that is nivolumab.

Each drug/agent included in the combination can be formulated separately for use in combination. The drugs are said to be used “in combination” when the effect of one drug is used to augment the effect of the other, in a recipient of both drugs.

In this approach, each drug is provided in a dosage form comprising a pharmaceutically acceptable carrier, and in a therapeutically effective amount. As used herein, “pharmaceutically acceptable carrier” means any and all solvents, dispersion media, coatings, antibacterial and antifungal agents, isotonic and absorption delaying agents, and the like that are physiologically compatible and useful in the art of protein/antibody formulation. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent. The SIRPαFc fusion and the protein-based PD-1 blockade drug are formulated using practises standard in the art of therapeutic protein formulation. Solutions that are suitable for intravenous administration, such as by injection or infusion, are particularly useful.

Sterile injectable solutions can be prepared by incorporating the active compound in the required amount in an appropriate solvent with one or a combination of ingredients noted above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation are vacuum drying and freeze-drying (lyophilization) that yield a powder of the active ingredient plus any additional desired ingredient from a previously sterile-filtered solution thereof.

As used herein, “effective amount” refers to an amount effective, at dosages and for a particular period of time necessary, to achieve the desired therapeutic result. A therapeutically effective amount of each drug in the combination may vary according to factors such as the disease state, age, sex, and weight of the individual, and the ability of the drug to elicit a desired response in the recipient. A therapeutically effective amount is also one in which any toxic or detrimental effects of the pharmacological agent are outweighed by the therapeutically beneficial effects.

The amount of active ingredient that can be combined with a carrier material to produce a single dosage form will vary depending upon the subject being treated, and the particular mode of administration. The amount of active ingredient required to produce a single, unit dosage form will generally be that amount of the composition that produces a therapeutic effect. Generally, out of one hundred percent, this amount will range from about 0.01 percent to about ninety-nine percent of active ingredient, preferably from about 0.1 percent to about 70 percent, e.g., from about 1 percent to about 30 percent of active ingredient in combination with a pharmaceutically acceptable carrier.

The SIRPαFc fusion protein and the PD-1 blockade drug, e.g. antibody, as well as the CTLA-4 inhibitor, may be administered to the subject through any of the routes established for protein delivery, in particular intravenous, intratumoural, intradermal and subcutaneous injection or infusion, or by nasal or pulmonary administration.

In some embodiments, variants of the polypeptides (or peptides, proteins, antibodies, and the like) described above are contemplated. For example, in some embodiments, the invention is practiced with a variant having at least 90%, at least 91%, at least 92%, at least 93%, at least 94%, at least 95%, at least 96%, at least 97%, at least 98%, or at least 99% amino acid sequence identity with respect to a reference polypeptide or sequence described herein. In some embodiments, the sequences differ only by conservative amino acid substitutions. The variants retain the relevant biological activities of the reference sequence described herein, such as CD47 binding (SIRPα variants), effector function (for Fc variants), or checkpoint inhibitor activity (for T cell checkpoint inhibitors).

The drugs in the present combination can be administered sequentially or, essentially at the same time. That is, T cell checkpoint inhibitor/s can be given before or after administration of SIRPαFc. It is desirable that the effects of the drugs overlap in the patient, and preferred that the drug substance or active metabolites are present at the same time in the recipient. Thus, a subject undergoing treatment can be a subject that has already received one of the combination drugs, such as SIRPαFc, and this subject is then treated with the other of the combination drugs. In the alternative, the subject can be one who has been treated with the T cell checkpoint inhibitor/s, and is then treated with the SIRPαFc.

A drug composition can be administered via one or more routes of administration using one or more of a variety of methods known in the art. As will be appreciated by the skilled artisan, the route and/or mode of administration will vary depending upon the desired results. Preferred routes of administration for fusion proteins of the invention include intravenous, intramuscular, intradermal, intraperitoneal, intratumoural, subcutaneous, spinal or other parenteral routes for administration, for example by injection or infusion. The phrase “parenteral administration” that includes infusion and injection such as intravenous, intramuscular, intraarterial, intrathecal, intracapsular, intraorbital, intracardiac, intradermal, intraperitoneal, transtracheal, intratumoural, subcutaneous, subcuticular, intraarticular, subcapsular, subarachnoid, intraspinal, epidural and intrasternal.

Alternatively, a fusion protein of the invention can be administered via a non-parenteral route, such as orally or by instillation or by a topical, epidermal or mucosal route of administration, for example, intranasally, orally, vaginally, rectally or sublingually.

Dosing regimens are adjusted to provide the optimum desired response (e.g., a therapeutic response). For example, a single bolus of each drug may be administered, or several divided doses may be administered over time or the dose may be proportionally reduced or increased as indicated by the therapeutic situation. It is especially advantageous to formulate parenteral compositions in unit dosage form for ease of administration and uniformity of dosage. “Unit dosage form” as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

The drugs can be formulated in combination, so that the combination can be introduced to the recipient in one administration, e.g., one injection or one infusion bag. In another embodiment, the drugs are formulated separately for separate administration in a combination therapy regimen.

For administration, the dose for each drug will be within the range from about 0.0001 to 100 mg/kg, and more usually 0.01 to 5 mg/kg, of the host body weight. For example dosages can be 0.1 mg/kg body weight, 0.2 mg/kg body weight, 0.3 mg/kg body weight, 1 mg/kg body weight, 3 mg/kg body weight, 5 mg/kg body weight or 10 mg/kg body weight or within the range of 1-10 mg/kg. Unit dosage forms, a drug will comprise from 1-500 mgs of drug, such as 1, 2, 3, 4 5, 10 25, 50, 100, 200, 250, and 500 mgs/dose. The two drugs can be administered in roughly equimolar amounts (+/−10%). An exemplary treatment regimen entails administration once per week, once every two weeks, once every three weeks, once every four weeks, once a month, once every 3 months or once every three to 6 months. Preferred dosage regimens for the drug combination of the invention include 1 mg/kg body weight or 3 mg/kg body weight via intravenous administration, with the drugs each being given simultaneously using one of the following dosing schedules; (i) every four weeks for six dosages, then every three months; (ii) every three weeks; (iii) 3 mg/kg body weight once followed by 1 mg/kg body weight every three weeks. In some methods, dosage is adjusted to achieve a plasma fusion protein concentration of about 1-1,000 ug/ml and in some methods about 25-300 ug/ml.

In embodiments, a subject is treated using a dosing regimen that includes SIRPαFc drug of SEQ ID NO: 8 or NO: 9 at 0.1 mg/kg weekly (or 0.2 mg/kg weekly, or 0.3 mg/kg weekly) and nivolumab at about 3 mg/kg every 2 weeks. Ipilimumab can also be integrated with dosing as approved when used with nivolumab. The SIRPαFc protein displays negligible binding to red blood cells. There is accordingly no need to account for an RBC “sink” when dosing with the drug combination. Relative to other CD47 blockade drugs that are bound by RBCs, it is estimated that the present SIRPαFc fusion can be effective at doses that are less than half the doses required for drugs that become RBC-bound, such as CD47 antibodies. Moreover, the SIRPα-Fc fusion protein is a dedicated antagonist of the SIRPα-mediated signal, as it displays negligible CD47 agonism when binding thereto. There is accordingly no need, when establishing medically useful unit dosing regimens, to account for any stimulation induced by the drug.

Each drug in the combination can also be administered as a sustained release formulation, in which case less frequent administration is required. Dosage and frequency vary depending on the half-life of the fusion protein in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient show partial or complete amelioration of symptoms of disease. Thereafter, the patient can be treated using a prophylactic regimen.

The drug combination is useful to treat a variety of CD47+ disease cells. These include particularly CD47+ cancer cells, including liquid and solid tumours. Solid tumours can be treated with the present drug combination, to reduce the size, number or growth rate thereof and to control growth of cancer stem cells. Such solid tumours include CD47+ tumours in melanoma, bladder, brain, breast, lung, colon, ovary, prostate, liver, skin and other tissues as well. In one embodiment, the drug combination can used to inhibit the growth or proliferation of hematological cancers. As used herein, “hematological cancer” refers to a cancer of the blood, and includes leukemia, lymphoma and myeloma among others. “Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML); chronic lymphocytic leukemia (CLL); chronic myelogenous leukemia (CML); myeloproliferative disorder/neoplasm (MPDS); and myelodysplastic syndrome. “Lymphoma” may refer to a Hodgkin's lymphoma, both indolent and aggressive non-Hodgkin's lymphoma, Burkitt's lymphoma, cutaneous T cell lymphoma, peripheral T cell lymphoma and follicular lymphoma (small cell and large cell), among others. Myeloma may refer to multiple myeloma (MM), giant cell myeloma, heavy-chain myeloma, and light chain or Bence-Jones myeloma.

In some embodiments, the hematological cancer treated with the drug combination is a CD47+ leukemia, preferably selected from acute lymphocytic leukemia, acute myeloid leukemia, chronic lymphocytic leukemia, chronic myelogenous leukemia, and myelodysplastic syndrome, preferably, human acute myeloid leukemia.

In other embodiments, the hematological cancer treated with the SIRPαFc protein is a CD47+ lymphoma or myeloma selected from Hodgkin's lymphoma, both indolent and aggressive non-Hodgkin's lymphoma, Burkitt's lymphoma, follicular lymphoma (small cell and large cell), multiple myeloma (MM), giant cell myeloma, heavy-chain myeloma, and light chain or Bence-Jones myeloma as well as leimyosarcoma.

In still other embodiments, the present drug combination is used for the treatment of non-small cell lung cancer, renal cancer, bladder cancer, head and neck squamous cell carcinoma, Merkel cell skin cancer, esophageal cancer, pancreatic cancer, hepatocellular carcinoma, gastric cancer, breast cancer and ovarian cancer. In specific embodiments, when the T cell checkpoint inhibitor is nivolumab, the treated cancer is one of melanoma, glioblastoma, metastatic non-small cell lung cancer, renal cell carcinoma, Hodgkin's lymphoma, head and neck cancer, urothelial carcinoma, colorectal cancer, and hepatocellular carcinoma. When the T cell checkpoint inhibitor is pembrolizumab, the treated cancer is one of melanoma, metastatic non-small cell lung cancer, head and neck cancer, Hodgkin's lymphoma, urothelial carcinoma and gastric cancer. In another specific embodiment, the target cancer is melanoma when the T cell checkpoint inhibitor is ipilimumab. In another specific embodiment, the target cancer is glioblastoma when the T cell checkpoint inhibitor is a PD-1 inhibitor.

In a specific embodiment, the subject receiving treatment is afflicted with Hodgkin's lymphoma, and the treatment comprises 0.1-0.3 mg/kg weekly of a SIRPαFc drug comprising SEQ ID NO: 8 or NO: 9, in combination with nivolumab at 3 mg/kg every 2 weeks. In another specific embodiment, this combination therapy is used for the treatment of subjects afflicted with melanoma.

The combination therapy, comprising CD47 blockade via SIRPαFc, and T-cell checkpoint inhibition such as by PD-1 blockade and/or CTLA-4 blockade, can also be exploited together with any other agent or modality useful in the treatment of the targeted indication, such as surgery as in adjuvant therapy, or with additional chemotherapy as in neoadjuvant therapy.

In a particular embodiment, when treatment involves the combination of ipilimumab and nivolumab, together with SIRPαFc, the drugs can be given concurrently (SIRPαFc given IV 1×/week or IT 3×/week). The recommended dose of nivolumab is 1 mg/kg administered as an intravenous infusion over 60 minutes, followed by ipilimumab 3 mg/kg on the same day, every 3 weeks for 4 doses. The recommended subsequent dose of nivolumab, as a single agent, is 240 mg administered as an intravenous infusion over 60 minutes every 2 weeks until disease progression or unacceptable toxicity.

### EXAMPLE 1

Female C57BL/6 mice were eight weeks old with a body weight (BW) range of 19.1 to 25.5 grams on Day 1 of the study. The animals were fed ad libitum water (reverse osmosis, 1 ppm Cl), and NIH 31 Modified and Irradiated Lab Diet® consisting of 18.0% crude protein, 5.0% crude fat, and 5.0% crude fiber. The mice were housed on irradiated Enrich-o'cobs™ Laboratory Animal Bedding in static micro isolators on a 12-hour light cycle at 20-22° C. (68-72° F.) and 40-60% humidity. CR Discovery Services specifically complies with the recommendations of the Guide for Care and Use of Laboratory Animals with respect to restraint, husbandry, surgical procedures, feed and fluid regulation, and veterinary care.

### Blockade Drugs

Trillium Therapeutics, Inc. provided pre-formulated CD47 blockade drugs and controls, as murine forms of soluble SIRPα designated (1) control Fc {mouse IgG2aFc region (hinge-CH2-CH3)}, and (2) mouse SIRPαFc {comprising the N-terminal domain of mouse SIRPA (NOD strain) fused to a wild type mouse IgG2a domain (hinge CH2-CH3)} which were stored at −80° C. until use. Mouse protein was used because human SIRPαFc proteins do not cross react with mouse CD47 target. PD-1 blockade drug was provided as anti-PD-1 antibody (Clone RMP1-14, Lot No. 5792/0915, 6.54 mg/mL) purchased from Bio X Cell by CR Discovery and stored at 4° C. upon receipt. The test drugs were formulated in sterile PBS and blinded during testing. On each dosing day, one vial of each agent was thawed and used for dosing at 0.2 mL (100 ug) per mouse. Each antibody dosing solution was prepared by diluting aliquots of the stock to 0.5 mg/mL in sterile PBS. Formulated antibody drugs were purchased.

### Tumor Cell Culture

MC38 murine colon carcinoma cells were grown to mid-log phase in DMEM medium containing 10% fetal bovine serum. The tumor cells were cultured in tissue culture flasks in a humidified incubator at 37° C., in an atmosphere of 5% CO2 and 95% air. On the day of tumor implant, MC38 cells were harvested during exponential growth and re-suspended in phosphate buffered saline (PBS) at a concentration of 5×10⁶ cells/mL. Tumors were initiated by subcutaneously implanting 1×10⁶ MC38 tumor cells (0.1 mL suspension) into the right flank of each test animal. Tumors were measured in two dimensions using calipers, and volume was calculated using the formula:

$$Tumor Volume ( {mm}^{3} ) = \frac{{w}^{2} \times l}{2}$$

where, w=width and l=length, in mm, of the tumor. Tumor weight can be estimated with the assumption that 1 mg is equivalent to 1 mm³ of tumor volume.

### Treatment

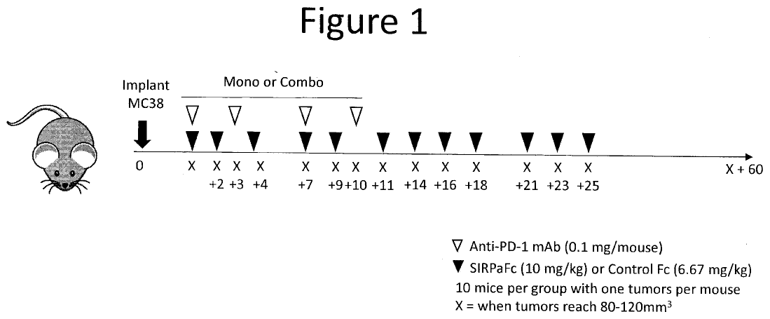

Following implant, tumors were monitored until they reached an average size of 80-120 mm³. Mice were sorted into five groups (n=10), and dosing was initiated. All agents were delivered intraperitoneally (i.p.). As shown in FIG. 1, Groups 1 and 2 received SIRPαFc at 200 μg/animal or Control Fc at 133 μg/animal three times per week for four weeks (tiwk×4), respectively. Group 3 received anti-PD-1 at 100 μg/animal, twice weekly for two weeks (biwk×2). Groups 4 and 5 received SIRPαFc or Control Fc, respectively, in combination with anti-PD-1 on the regimens described above.

### Endpoint Analysis

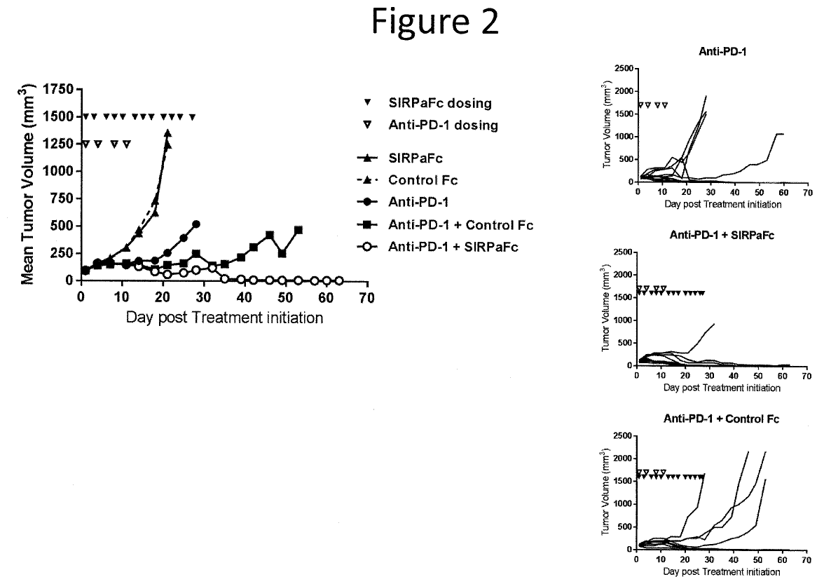

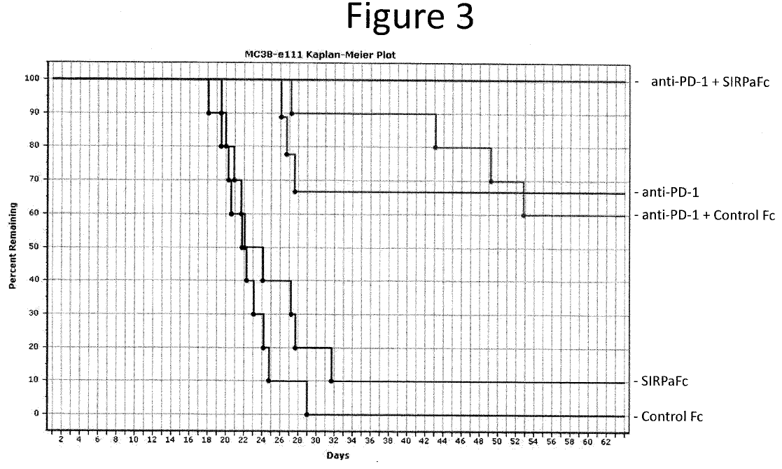

Tumors were measured using calipers twice per week. Animals were monitored individually, and each mouse was euthanized when its tumor reached the endpoint volume of 1500 mm³ or on the final day, whichever came first. Animals that exited the study for tumor volume endpoint were documented as euthanized for tumor progression (TP), with the date of euthanasia. The time to endpoint (TTE) for analysis was calculated for each mouse by the following equation:

$$T T E = \frac{{log}_{10} ( endpoint volume ) - b}{m}$$

where TTE is expressed in days, endpoint volume is expressed in mm³, b is the intercept, and m is the slope of the line obtained by linear regression of a log-transformed tumor growth data set. The data set consisted of the first observation that exceeded the endpoint volume used in analysis and the three consecutive observations that immediately preceded the attainment of this endpoint volume. The calculated TTE is usually less than the TP date, the day on which the animal was euthanized for tumor size. Animals with tumors that did not reach the endpoint volume were assigned a TTE value equal to the last day of the study. In instances in which the log-transformed calculated TTE preceded the day prior to reaching endpoint or exceeded the day of reaching tumor volume endpoint, a linear interpolation was performed to approximate the TTE. As shown in the FIGS. 1-3, SIRPαFc alone had no effect on MC38, suggesting there were not enough macrophages infiltrating the MC38 tumour, or that this tumour grows too quickly to be controlled.

### EXAMPLE 2

In this study, a human tumor cell line, Jurkat, was transfected to express human cytomegalovirus (CMV) pp65 antigen. The CMV pp65 antigen is used as a surrogate tumor antigen.

Cytomegalovirus (CMV)-specific CD8+ T cells were used as a source of surrogate tumor antigen specific T cells. These CMV-specific CD8+ T cells were isolated from the blood of a healthy HLA-A2+ donor and were expanded over a 14-day period by co-culturing with autologous mature CMV peptide pp65-pulsed dendritic cells in the presence of IL-7 and IL-15 according to a standard protocol (Wolf and Greenberg, Nat Protoc. 9(4) 2014).

Monocyte derived macrophages were generated by culturing blood monocytes in M-CSF for 9 days, followed by priming with IFNγ for 24 hours. These IFNγ primed macrophages were subsequently co-cultured with CMV pp65-transfected Jurkat to allow phagocytosis to happen in the presence of 1 uM SIRPαFc. At 24 hour of the co-culture, pp65 antigen was confirmed to be presented on the surface of macrophages by flow cytometry (data not shown). At this time, expanded CMV-specific autologous CD8+ T cells were added to the macrophages.

Degranulation of CMV-specific CD8+ T cells was assessed by the addition of FITC-conjugated anti-CD107a/b mAbs for 5 hours and subsequent analysis by flow cytometry. Intracellular cytokine production by CMV-specific CD8+ T cells was assessed by permeabilization and staining with anti-TNFα and anti-IFNγ mAbs, followed by flow cytometry. CMV-specific CD8+ T cells were identified by concurrent tetramer staining. Ipilimumab and Nivolumab were added at 10 ug/mL where indicated.

### Summary of the Results

(A) The triple combination, Nivolumab and Ipilimumab with SIRPαFc, resulted in a statistically significant increase in tumor specific CD8 T cell activation compared to SIRPαFc alone, as measured by the percent of tumor specific CD8+ T cells that are CD107a/b+, a marker of degranulation.

(B) The two combinations, Nivolumab with SIRPαFc and Ipilimumab with SIRPαFc, resulted in statistical significant increases in the percentage of TNFα+ IFNγ+ of tumor specific CD8 T cells compared to SIRPαFc treatment alone. The triple combination, nivolumab and ipilimumab with SIRPαFc, further increased the percentage of TNFα+ IFNγ+ of tumor specific CD8 effector T cells compared to the dual combinations and SIRPαFc mono-treatment.

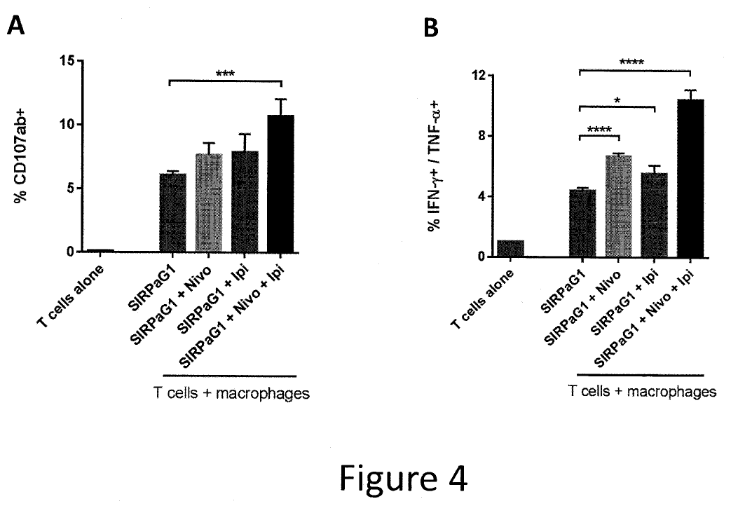

It is thus shown that statistically significant improvements in the anti-cancer effect of SIRPαFc are obtained when treatment is combined with the PD-1 inhibitor nivolumab (increased by 50% as shown in FIG. 4B), or with the CTLA-4 inhibitor ipilimumab (increased by 26% as shown in FIG. 4B), or with both PD-1 inhibitor and CTLA-4 inhibitor (increased by 136% and by 76% as shown in FIGS. 4B and 4A, respectively).

Collectively, the combination of either nivolumab or ipilimumab with SIRPαFc and the triple combination, nivolumab and ipilimumab with SIRPαFc, resulted in increased activation and effector function of tumor specific CD8+ T cells compared to SIRPαFc treatment alone.

### EXAMPLE 3

Combination Therapy in Subjects with Relapsed and Refractory Percutaneously-Accessible Solid Tumors or Mycosis Fungoides

Following are protocols for demonstrating efficacy of a SIRPαFc—checkpoint inhibitor combination in human subjects with solid tumors or mycosis fungoides in need of treatment.

SIRPαFc in combination with a programmed death-1 (PD-1) or programmed death-ligand-1 (PD-L1) inhibitors (such as nivolumab, pembrolizumab, durvalumab, avelumab, or atezolizumab) are administered to a subject with cancer on Day 1. In some variations, subjects have a cancer diagnosis for which a PD-1/PD-L1 inhibitor is approved by the FDA, such as melanoma, head and neck cancer, lung cancer, bladder cancer, urothelial cancer, colorectal cancer, breast cancer, and kidney cancer. In some variations, the subjects have mycosis fungoides.

A starting dose of 1 mg is selected of SIRPαFc (SEQ ID NO: 8, for instance), a SIRPαFc fusion protein that consists of the CD47-binding domain of human SIRPα linked to the Fc region of a human immunoglobulin (IgG1) for intratumoral injection. This dose ensures that the total systemic dose, even at 100% theoretical bioavailability, would be not more than about 0.05 mg/kg or 3 mg in a 60 kg subject. Intratumoral dosing can escalate up to 10 mg, 3 times per week, for 2 weeks. This dose is anticipated to achieve a very high local CD47 saturation, while corresponding approximately to the daily dose that can be received by IV administration (0.3 mg/kg or 18 mg in a 60 kg subject).

The safety of the above dose and dosing regimen was also supported by a non-human primate toxicity study that showed that subcutaneous administration of SIRPαFc (monotherapy) at doses of 0.5 mg/kg, administered 6 times over a 2-week period, and 1.5 mg/kg, administered twice over a 2-week period, produced no adverse dermal effects.

Expected hematology changes were observed with no meaningful biological difference noted between the dose levels. The highest non-severely toxic dose (HNSTD) for this study was 0.5 mg/kg, administered 6 times over a 2-week period, and 1.5 mg/kg, administered twice over a 2 week period.

Subjects will receive SIRPαFc in combination with one of the following PD-1/PD-L1 inhibitors administered IV on Day 1, according to the standard labeled dose and regimen: Nivolumab (OPDIVO®, Bristol Myers Squibb Company); Pembrolizumab (KEYTRUDA®, Merck and Co., Inc.); Durvalumab (IMFINZI™, AstraZeneca Pharmaceuticals LP); Avelumab (BAVENCIO®, EMD Serono, Inc.; and Pfizer Inc.); and Atezolizumab (TECENTRIQ®, Genentech, Inc. and Hoffman-La Roche Ltd.).

If the PD-1/PD-L1 inhibitor is given on the same day as SIRPαFc, at least 60 minutes should elapse between completion of the PD-1/PD-L1 inhibitor infusion and injection of SIRPαFc. The PD-1/PD-L1 inhibitor may also be given the day before SIRPαFc injection. Any PD-1/PD-L1 inhibitor infusion-related reactions should be Grade 2 or lower and have fully resolved to initiate SIRPαFc injection on the same day.

Efficacy is evaluated with respect to tumor volume, side effects, progression-free survival, overall survival, or other standard parameters, compared to subjects that receive single agent (SIRPαFc alone or checkpoint inhibitor alone).

Subjects who are eligible to receive continuation therapy following completion of their initial induction therapy may, at the discretion of the oncologist, receive additional weekly injections with SIRPαFc. The combination therapy will be continued using the standard dose and dosing regimen (see above).

### EXAMPLE 4

Phase 1a/1b Dose Escalation and Expansion Trial of SIRPαFc in Subjects with Relapsed or Refractory Hematologic Malignancies and Selected Solid Tumors

SIRPαFc plus Nivolumab subjects receive a starting dose of 0.1 mg/kg/week SIRPαFc in combination with nivolumab, dose per current FDA approved package insert, given every 2 weeks (1 cycle). Subjects who have unacceptable toxicity to nivolumab may continue to receive SIRPαFc as a single agent. If nivolumab is given on the same day as SIRPαFc, at least 60 minutes must elapse between completion of the nivolumab infusion and initiation of the SIRPαFc infusion. Nivolumab may also be given the day before SIRPαFc infusion.

Efficacy is evaluated with respect to cancer burden, side effects, progression-free survival, overall survival, or other standard parameters, compared to subjects that receive single agent (SIRPαFc alone or checkpoint inhibitor alone).

We claim:

1. A method for treating a subject presenting with CD47+ disease cells, comprising administering to the subject a T cell checkpoint inhibitor and a CD47 blockade drug.

2. The use of a T cell checkpoint inhibitor and a CD47 blockade drug to treat a subject presenting with CD47+ disease cells.

3. A T cell checkpoint inhibitor for use in treating CD47+ disease cells by co-administration with a CD47 blockade drug.

4. A CD47 blockade drug for use in treating CD47+ disease cells by co-administration with a T cell checkpoint inhibitor.

5. The use of a T cell checkpoint inhibitor and a CD47 blockade drug in the manufacture of a medicament for treating CD47+ disease cells.

6. The use of a T cell checkpoint inhibitor in the manufacture of a medicament for treating CD47+ disease cells by co-administration with a CD47 blockade drug.

7. The use of a CD47 blockade drug in the manufacture of a medicament for treating CD47+ disease cells by co-administration with a T cell checkpoint inhibitor.

8. A product comprising a T cell checkpoint inhibitor and a CD47 blockade drug as a combined preparation for simultaneous, separate, or sequential use in the treatment of CD47+ disease cells.

9. A composition comprising a T cell checkpoint inhibitor, a CD47 blockade drug, and a pharmaceutically acceptable carrier.

10. The method, use, product, or composition according to any one of claims 1-9, wherein the CD47+ disease cells comprise CD47+ cancer cells.

11. The method, use, product, or composition according to any one of claims 1-9, wherein the T cell checkpoint inhibitor comprises a PD-1 blockade drug.

12. The method, use, product, or composition according to claim 11, wherein the PD-1 blockade drug comprises an agent that binds PD-1.

13. The method, use, product, or composition according to claim 12, wherein the PD-1 blockade drug comprises nivolumab.

14. The method, use, product, or composition according to any one of claims 1-13, wherein the PD-1 blockade drug comprises an agent that binds PD-L1 or PD-L2.

15. The method, use, product, or composition according to claim 14, wherein the PD-1 blockade drug comprises a PD-L1 binding agent.

16. The method, use, product, or composition according to claim 15, wherein the PD-L1 binding agent comprises a member selected from durvalumab, atezolizumab, avelumab and the IgG4 antibody designated BMS-936559/MDX1105

17. The method, use, product, or composition according to any one of claims 1-16, wherein the T cell checkpoint inhibitor comprises a CTLA4 inhibitor.

18. The method, use, product, or composition according to claim 17, wherein the CTLA4 inhibitor comprises a CTLA4 antibody.

19. The method, use, product, or composition according to claim 18, wherein the CTLA4 antibody comprises ipilimumab or tremelimumab.

20. The method, use, product, or composition according to any one of claims 1-19, wherein the CD47 blockade drug comprises an Fc fusion protein comprising a soluble CD47-binding region of human SIRPα fused to an Fc region of an antibody.

21. The method, use, product, or composition according to claim 20, wherein the Fc fusion protein comprising soluble SIRPα comprises the amino acid sequence of SEQ ID NO: 8.

22. The method, use, product, or composition according to claim 20, wherein the Fc fusion protein comprising soluble SIRPα comprises the amino acid sequence of SEQ ID NO: 9.

23. The method, use, product, or composition according to any one of claims 1-19, wherein the CD47 blockade drug comprises soluble SIRPα having one or more amino acid substitutions selected from L⁴V/I, V⁶I/L, A²¹V, V²⁷I/L, I³¹T/S/F, E⁴⁷V/L, K⁵³R, E⁵⁴Q, H⁵⁶P/R, S66T/G, K⁶⁸R, V⁹²I, F⁹⁴V/L, V⁶³I, and F¹⁰³V.

24. The method, use, product, or composition according to any one of claims 1-23, wherein the T cell checkpoint inhibitor comprises a combination of nivolumab and ipilimumab.

25. The method, use, product, or composition according to any one of claims 1-24, wherein the CD47+ disease cells comprise blood cancer cells or solid tumour cancer cells.

26. The method, use, product, or composition according to claim 25, wherein the CD47+ disease cells are cells of a cancer type selected from acute lymphocytic leukemia (ALL); acute myeloid leukemia (AML); chronic lymphocytic leukemia (CLL); chronic myelogenous leukemia (CML); myeloproliferative disorder/neoplasm (MPDS); and myelodysplastic syndrome.

27. The method, use, product, or composition according to claim 25, wherein the cancer is a lymphoma selected from a Hodgkin's lymphoma, both indolent and aggressive non-Hodgkin's lymphoma, Burkitt's lymphoma, and follicular lymphoma (small cell and large cell).

28. The method, use, product, or composition according to claim 25, wherein the cancer is a myeloma selected from multiple myeloma (MM), giant cell myeloma, heavy-chain myeloma, and light chain or Bence-Jones myeloma.

29. The method, use, product, or composition according to claim 25, wherein the cancer is a melanoma.

30. The method, use, product, or composition according to claim 25, wherein the cancer is AML, myelodysplastic syndrome, CLL, Hodgkin lymphoma, indolent B cell lymphoma, aggressive B cell lymphoma, T cell lymphoma, multiple myeloma, myeloproliferative neoplasms, or CD20+ lymphoma.

31. The method, use, product, or composition according to claim 25, wherein the cancer is selected from non-small cell lung cancer, renal cancer, bladder cancer, head and neck squamous cell carcinoma, Merkel cell skin cancer, esophageal cancer, pancreatic cancer, hepatocellular carcinoma, glioblastoma, gastric cancer, breast cancer and ovarian cancer.

32. The method, use, product, or composition according to claim 25, wherein the cancer is selected from melanoma, metastatic non-small cell lung cancer, head and neck cancer, Hodgkin's lymphoma, urothelial carcinoma and gastric cancer.

33. The method, use, product, or composition according to any one of claims 1-32, wherein the T cell checkpoint inhibitor and the CD47 blockade drug are present or used in synergistically effective amounts.

34. A pharmaceutical combination of anti-cancer agents, comprising a SIRPαFc and a T cell checkpoint inhibitor effective to enhance SIRPαFc-mediated depletion of CD47+ disease cells.

35. The use of the combination according to claim 34, for the treatment of a subject presenting with CD47+ cancer cells.

36. The use according to claim 35, wherein the CD47+ disease cells are CD47+ cancer cells.

37. A kit comprising at least one of SIRPαFc and a T cell checkpoint inhibitor, and instructions teaching the use thereof according to the method, use, product, or composition of any one of claims 1-33.
